(19) United States
(12) Patent Application Publication
(10) Pub. No.: US 2002/0146745 A1
(43) Pub. Date: Oct. 10, 2002

(54) Methods and reagents for multiplexed analyte capture, surface array self-assembly, and analysis of complex biological samples

(75) Inventors: Michael J. Natan, Los Altos, CA (US); Howard Schulman, Palo Alto, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SURROMED, INC., Mountain View, CA

(21) Appl. No.: 10/115,863

(22) Filed: Apr. 3, 2002

Related U.S. Application Data
(60) Provisional application No. 60/281,228, filed on Apr. 3, 2001.
(60) Provisional application No. 60/281,041, filed on Apr. 3, 2001.

(51) Int. Cl. G01N 33/53

(52) U.S. Cl. 435/7.1

(57) ABSTRACT

Bifunctional capture probes used for multiplexed assays consist of particles bearing analyte-binding moieties and pairing oligonucleotides, which hybridize to an array of surface-bound capture oligonucleotides. Capture probes are combined with a sample containing analytes of interest, extracted from the sample, and then exposed to the oligonucleotide array. Based on their pairing oligonucleotide sequences, the capture probes self-assemble at particular array locations. Bound analytes are then detected using a method, such as mass spectrometry, that can be directed toward particular array locations. Because any number and combination of capture probes can be employed, the method is flexible and able to detect analytes at very low concentrations. Additionally, the method provides the ease of detection associated with position-addressable arrays.

### CROSS-REFERENCE TO RELATED APPLICATIONS

This application claims the benefit of U.S. Provisional Application No. 60/281,228, “Mass Spectrometric Analysis of Complex Biological Samples,” filed Apr. 3, 2001, and U.S. Provisional Application No. 60/281,041, “Combinatorial Separation Platform for Comprehensive Molecular Analysis of Functional Cancer Biomarkers,” filed Apr. 3, 2001, both of which are incorporated herein by reference.

### FIELD OF THE INVENTION

The present invention relates generally to separation, detection, and identification of multiple analytes in complex biological samples. More particularly, it relates to a method of capturing analytes from solution using capture probes that self-assemble at defined locations on an array for subsequent analysis by methods such as mass spectrometry.

### BACKGROUND OF THE INVENTION

Discovering and identifying proteins, metabolites, and other molecules of interest in complex biological samples requires effective separation and detection techniques. Because of the enormous number, diverse biophysical properties, and large variation in concentrations of the components, it is difficult to find methods that perform well for a broad variety of biological molecules. For studies in which broad biological profiling is performed, it is highly desirable to be able to detect a large number and variety of biological molecules simultaneously in small sample volumes.

Bioanalytical methods often make use of very specific and high-affinity interactions between certain biological binding pairs such as antigens and antibodies, ligands and receptors, and complementary oligonucleotide sequences. By providing one member of the pair, the other member can be extracted from a sample for further analysis. Typically, a set of complementary binding moieties to analytes of interest is affixed to a solid surface, and the sample is contacted with the surface, causing analytes to bind to the surface-bound capture agents. Detection of bound analyte is enabled by radioactive or fluorescent tags bound to the analyte. For example, microarrays of oligonucleotide (e.g., DNA) probes are in widespread use for gene expression monitoring and diagnostic applications. Single-stranded oligonucleotide sequences are immobilized on a surface and can hybridize to complementary oligonucleotide sequences in the sample. Spatially addressed arrays, in which thousands of different oligonucleotide probes are immobilized to different locations of the surface, permit target nucleic acid molecules to be sequenced. The target molecule is tagged with, e.g., a fluorescent label and contacted with the surface, and the locations to which it binds detected. Spatially-addressed oligonucleotide arrays can also be used for multiplexed assays, in which multiple analytes bind to multiple locations on the array.

Analogous arrays have been prepared for studying proteins and their interactions; in these arrays, immobilized capture reagents are antibodies or other agents that bind proteins with sufficient affinity. For example, proteins have been immobilized to glass microscope slides and shown to interact specifically with other proteins or small molecules in solution, as described in G. MacBeath and S. L. Schreiber, “Printing Proteins as Microarrays for High-Throughput Function Determination,” Science 189:1760-1763 (2000). Interactions with surface-bound proteins are detected via fluorescent tags attached to molecules in solution. U.S. Pat. No. 6,329,209, issued to Wagner et al., provides arrays of protein-capture agents useful for simultaneous detection of a plurality of protein products.

Other methods for detecting analytes bound to surface-immobilized capture agents have been provided. For example, U.S. Pat. No. 6,020,208, issued to Hutchens et al., discloses a method for detecting analytes by capturing the analytes on a probe containing an immobilized affinity reagent. The probe is then presented for analysis by a mass spectrometric technique incorporating matrix-assisted laser desorption ionization (MALDI) or surface-enhanced laser desorption ionization (SELDI). A related technique is described in U.S. Patent Application Publication No. US 2001/0019829, which discloses multiplexed immunoassays performed by affinity capture of antigens onto a solid surface followed by elution of the antigens and mass spectral analysis. Because mass spectrometry is an inherently multiplexed technique, there is no need to correlate captured antigens to bound probes in this method, and all bound antigens can be analyzed simultaneously.

Attaching proteins to surfaces is generally more difficult than attaching oligonucleotides to surfaces. U.S. Patent Application Publication No. US 2002/0028455 discloses an array of support-bound probes in which the analyte-binding moieties are covalently linked to oligonucleotides that hybridize to surface-bound oligonucleotides. Once formed, the surfaces can be used in any application requiring an array of support-bound probes such as proteins. Other methods also make use of an oligonucleotide-labeled antibody. For example, U.S. Pat. No. 6,110,687, issued to Nilsen, provides a method for detecting antigen-antibody binding by immobilizing antigens to a surface and contacting them with antibodies. The antigen-antibody complex is then combined with a secondary antibody linked to an oligonucleotide hybridized to a radioactively-labeled complementary oligonucleotide that may be part of a dendrimer.

The ability of complementary oligonucleotides to self-assemble has been exploited to create complex nanoscale structures; see, for example, S. -J. Park et al., “Directed Assembly of Periodic Materials from Protein and Oligonucleotide-Modified Nanoparticle Building Blocks,” Angew. Chem., Int. Ed. 40:2909-2912 (2001), incorporated herein by reference. Recently, it has been shown that nanoscale oligonucleotide-coated cylindrical particles bind to an array of complementary oligonucleotides, but not to other surface regions, as described in D. J. Pena et al., “Electrochemical Synthesis of Multi-Material Nanowires as Building Blocks for Functional Nanostructures,” MRS Symp. Proc. 636 (2001), incorporated herein by reference. DNA-directed immobilization has also been shown to be site-specific; covalent DNA-streptavidin conjugates were coupled to biotinylated enzymes and allowed to hybridize to complementary, surface-bound capture oligonucleotides, as described in C. M. Niemeyer et al., “DNA-Directed Immobilization: Efficient, Reversible, and Site-Selective Surface Biding of Proteins by Means of Covalent DNA-Streptavidin Conjugates,” Anal. Biochem. 268:54-63 (1999), incorporated herein by reference. Three different conjugates bound predominantly to their complementary oligonucleotides.

The broad utility of protein arrays (termed “protein chips”) ensures that they will play an important role in profiling the human proteome, a task that requires high-throughput protein expression profiling as well as analysis of protein function, interaction, and structure. However, protein chips suffer from two significant problems, lack of flexibility and limited dynamic range. Although a single chip can contain thousands of different protein-capture agents, the need to add or subtract a single protein from the assay necessitates construction of an entirely new chip. More problematic is the limited dynamic range. The binding of molecules to arrays can be described by the law of mass action, according to which the percentage of capture probes having bound analyte is a function of the analyte concentration and equilibrium dissociation constant. When the analyte concentration is very low or the dissociation constant high (low-affinity binding), a small fraction of the probes are occupied, making the analyte difficult to detect if an insufficient number of binding sites are provided. Additionally, analytes at high concentration saturate the capture agents, making accurate analyte quantification impossible. The spot size (surface area on which each type of capture probe is bound) of a protein chip is typically fixed and does not allow for different capture probes to be immobilized in different amounts based on the concentration of corresponding analyte. As a result, it is difficult to detect and accurately quantify analytes present at very high or very low concentrations using protein arrays. This problem is particularly pronounced for complex biological samples; for example, concentrations of different soluble proteins in blood can vary by more than six orders of magnitude in a single sample, and further variations are found between samples.

Rather than being immobilized on the surface of a single substrate, capture probes can be distributed in solution to bind analytes and then recovered and identified. Solution-based capture is much more flexible than array-based methods, because the number and selection of capture probes can be varied with each assay. It is also typically faster than array methods, which are limited by the diffusion of analytes to the surface of the array. Unlike methods using arrays of immobilized probes, methods incorporating capture probes in solution require an additional separation step to recover bound analyte. Depending upon the nature and size of the capture probes, they can be recovered by centrifugation, affinity capture, magnetic fields, or other methods. In affinity capture methods, the capture probe has, in addition to the binding moiety that captures the analyte, an additional binding moiety specific for a molecule fixed to a solid surface. For example, a common binding pair used is biotin-avidin or biotin-streptavidin; biotin in a capture probe binds to surface-bound streptavidin, and the remaining unbound solution can be washed away. For example, a method for separating proteins for mass spectrometric analysis is disclosed in PCT Published Application WO 00/11208. In this method, proteins are captured by protein-reactive reagents, bound to an affinity column and then subsequently eluted. Only a single type of affinity pair is provided.

In solution capture methods, because the array position is not available as a variable to identify the capture probe, additional techniques are incorporated to allow for significant multiplexing. For example, U.S. Pat. No. 5,981,180, issued to Chandler et al., provides a multiplexed analysis method in which latex beads bearing capture agents are impregnated with different ratios of fluorescent dyes that encode the identity of the capture probes. Each bead must be read individually using, e.g., a flow cytometer, to identify the capture agent and bound analyte. Bound analytes can be detected via fluorescently tagged secondary antibodies that bind to the analytes.

Multiplexed solution-based analyte detection methods therefore also suffer from a number of drawbacks. They require a potentially complicated detection system capable of decoding each particle to identify the capture probe. They do not allow for identification of unknown analytes. Recent developments in mass spectrometry have made it an important method for identifying unknown components of biological samples. In order to combine mass spectrometry with solution-based assays, numerous complicated steps are required to remove the bound analyte from each capture probe after it has been identified. Such methods would be difficult to automate for high-throughput analysis.

U.S. Patent Application Publication No. US 2001/0031469 discloses a method for detecting modified proteins and other molecules using tagged substrates that react with sample analytes. The tags are complementary to immobilized elements of an array (e.g., DNA or peptide nucleic acids); after reacting in solution, the substrates sort in a preordered fashion onto the array. Detection is by fluorescence, chemiluminescence, radioactive labeling, or other suitable methods. A similar analyte detection method is described in Y. Oku et al., “Development of oligonucleotide lateral-flow immunoassay for multi-parameter detection,” J. Immunol. Methods 258:73-84 (2001). In this method, antigens bind to a detection antibody and tagged antibody, and the resulting complex binds to a nitrocellulose surface via interaction between the antibody tag and surface-bound oligonucleotides. Rather than being in an array configuration, the surface-bound oligonucleotides are arranged as parallel stripes, and the sample is flowed in a direction perpendicular to the stripes. Because this method is designed for visual inspection, it cannot accommodate a large dynamic range or stripe density. Furthermore, lateral flow of sample across the oligonucleotide stripes means that the stripes extract their complementary oligonucleotides in a sequential manner, a less precise process than the equilibrium attained with chip arrays. Additionally, when a single antibody is conjugated to a single oligonucleotide, a large array surface area is required for accurate analyte quantification at high concentration.

There is still a need, therefore, for a method for simultaneously detecting multiple analytes in complex biological samples. There is a particular need for methods that are flexible, can be automated, and provide a large dynamic range.

### SUMMARY OF THE INVENTION

The present invention provides methods for detecting multiple analytes simultaneously in a sample suspected of containing the analytes. By combining the advantages of solution-based capture probes and surface-bound capture arrays, the invention provides a flexible method for detecting analytes at a wide range of concentrations in complex biological samples.

In one embodiment, the invention provides a method for detecting analytes containing three steps: contacting the sample with a plurality of capture probes, allowing the capture probes to self-assemble on an array of surface-bound capture moieties, preferably oligonucleotides, and detecting analytes bound to the self-assembled capture probes, preferably by mass spectrometry. Each capture probe consists of a particle, binding moieties (e.g., proteins) capable of binding to an analyte, and pairing moieties, preferably single- or double-stranded oligonucleotides, which are substantially complementary to one of the surface-bound capture oligonucleotides. The particle is preferably a cylindrical metal particle with dimensions of at most approximately 100 nm. Preferably, the binding moieties and pairing oligonucleotides are affixed to different segments of the particle.

When the capture probes are contacted with the array, the pairing oligonucleotides and capture oligonucleotides hybridize to form a binding complex. Preferably, different surface-bound capture oligonucleotides are located at particular or predetermined positions of the array. Different subsets of capture probes have different pairing oligonucleotides and binding moieties, so that particular binding moieties are directed to particular locations of the array. In an additional embodiment, a single binding moiety is capable of binding to a plurality of different analytes. If desired, the bound analytes, with or without their binding moieties, can be removed from the array before analysis.

In an alternative embodiment, the present invention provides a capture probe containing a particle having at least two segments and dimensions of at most approximately 100 nm. A plurality of binding moieties such as proteins are affixed to one of the segments, and a plurality of oligonucleotide sequences are fixed to one of the segments, preferably to a different segment. Preferably, the particle is a cylindrical metal particle. The particle can be made superparamagnetic by making at least one of the segments of a ferromagnetic material such as cobalt or nickel.

### BRIEF DESCRIPTION OF THE FIGURES

FIGS. 1A-1D are schematic drawings outlining a method of the present invention for analyte capture and detection.

FIGS. 2A-2B illustrate two embodiments of a capture probe used in the method of FIGS. 1A-1D.

FIG. 3 shows a preferred embodiment of the capture probe of FIG. 2A.

FIGS. 4A-4B are schematic drawings outlining a method for derivatizing particles to obtain the capture probe of FIG. 3.

FIG. 5 shows a microfluidic device for performing the method of FIGS. 1A-1D.

### DETAILED DESCRIPTION OF THE INVENTION

The present invention provides methods and reagents for multiplexed separation, detection, and identification of analytes in a complex sample such as a biological sample. Analyte extraction is performed by particle-based capture probes that are distributed in solution. After binding to analyte, the capture probes self-assemble onto particular locations of an array of surface-bound oligonucleotides for identification and analysis. The method therefore incorporates the flexibility and large dynamic range of solution-phase capture with the high multiplexing capabilities and ease of detection of spatially-addressed arrays. Because the capture probes are constructed from particles, they provide a large surface area for analyte capture without requiring a large array surface area. Additionally, the method is scalable to different sample sizes and analyte concentrations, can be automated easily, and is customizable to assays ranging from specific analyte detection to disease-specific or class-specific profiling to broad comprehensive profiling.

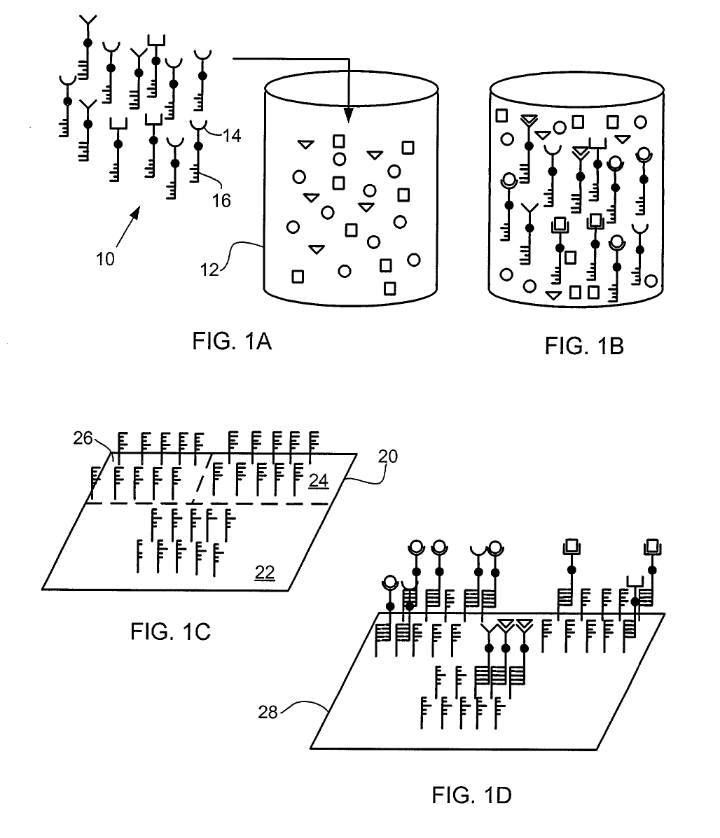

FIGS. 1A-1D schematically illustrate a method of the present invention for simultaneously detecting multiple analytes. Although only three analytes (represented by triangles, squares, and circles) are shown for clarity, hundreds or even thousands of analytes can be detected simultaneously using the present invention. It is to be understood that the phrases “detecting analytes” or “detection of analytes,” as used herein, refer to the process of assaying for analyte, and does not imply that bound analyte is found. For example, in a fluorescence-based detection process, detecting analyte describes the process of measuring emitted light over the appropriate wavelength range, even if there is no light emitted at that wavelength range.

Broadly, the invention has three main steps: analyte capture, capture probe self-assembly, and detection of captured analyte. In the first step, shown in FIG. 1A, a set of capture probes 10 is incubated with a sample 12 that potentially contains analytes to which the capture probes 10 can bind. The capture probe 10 can take many forms, but generally consists of a particle supporting a set of binding moieties 14, which are capable of binding to an analyte, and a set of pairing moieties 16, typically pairing oligonucleotides. Different analytes bind to the binding moieties of different capture probes, as shown in FIG. 1B. The capture probes 10 containing bound analyte can be recovered by centrifuging the mixture and removing the supernatant.

Next, the entire sample, or alternatively only recovered capture probes 10, is combined with a hybridization buffer and contacted with an array 20, shown in FIG. 1C, of different surface-bound complementary moieties, which are capable of interacting with the pairing moieties 16 of the capture probes 10 to form binding complexes. Typically, the array contains surface-bound capture oligonucleotides that are substantially complementary to the pairing oligonucleotides of the capture probes. In FIG. 1C, a region 22 contains capture oligonucleotides complementary to the pairing oligonucleotides linked to anti-triangle antibodies, a region 24 contains capture oligonucleotides complementary to the pairing oligonucleotides linked to anti-square antibodies, and a region 26 contains capture oligonucleotides complementary to the pairing oligonucleotides linked to anti-circle antibodies. When the capture probes 10 are incubated with the array 20, they self-assemble at the particular locations containing capture oligonucleotides with which their pairing oligonucleotides can hybridize, yielding the array 28 shown in FIG. 1D. Because the locations of particular capture oligonucleotides are predetermined, the identity of the analyte-binding moieties at each array position 22, 24, and 26 is known. The array 28 is then washed to remove salts and unbound capture probes or analyte. In the third step, the bound analytes are detected, preferably by mass spectrometry, which allows the identity of unknown analytes to be determined. Alternatively, the analytes can be detected using conventional detection methods for solid-phase assays (e.g., fluorescence). If desired, the analytes, with or without their capture probes, can be removed from the array for analysis.

Although FIGS. 1A-1D illustrate a multiplexed analyte detection method, the present invention can be used for capturing and detecting a single analyte. In this case, only a single type of capture probe is needed, and the array of surface-bound capture oligonucleotides contains oligonucleotides of identical sequence.

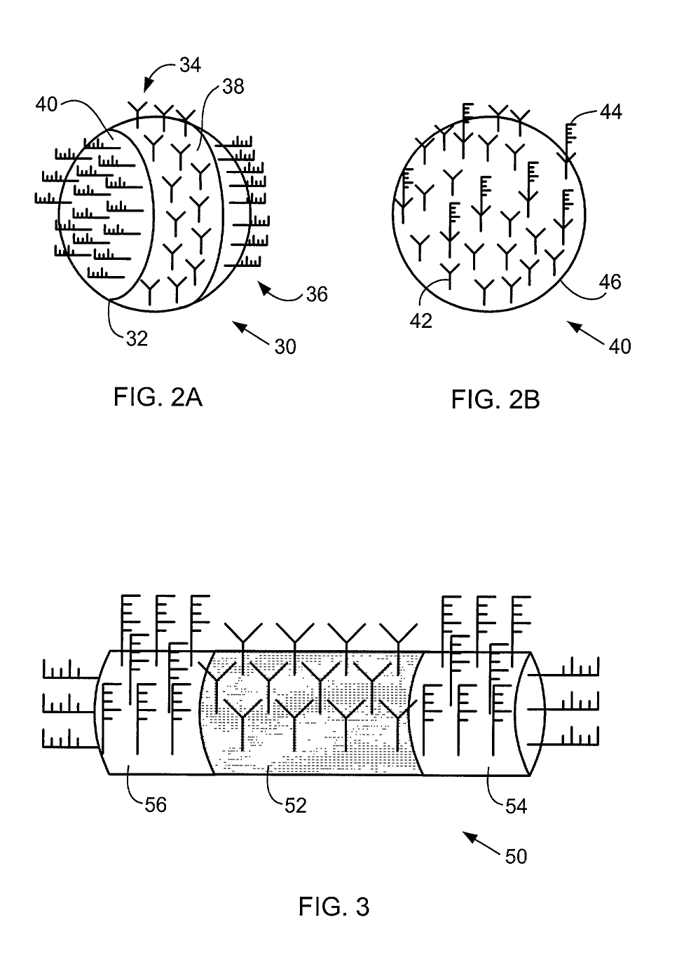

FIG. 2A shows a capture probe 30 of the present invention. The capture probe 30 consists of a small (micro- or nanoscale) particle 32 to which analyte-binding moieties 34 and pairing oligonucleotides 36 are affixed, preferably at discrete regions 38 and 40, respectively, of the particle surface. The discrete regions 38 and 40 may have different surface compositions that facilitate binding of the respective element only. However, the binding moieties 34 and pairing oligonucleotides 36 can instead be distributed evenly over the particle surface. Typically, hundreds of binding moieties 34 and pairing oligonucleotides 36 are attached to a single particle 32. The particle diameter (or other suitable measure, for non-spherical particles) ranges between approximately 10 nm and approximately 1 μm. Preferably, the particle diameter is between approximately 50 and 100 nm.

FIG. 2B shows an alternative embodiment of a capture probe. In a capture probe 40, only one of the binding moiety 42 and pairing oligonucleotide 44 is attached directly to the surface of a particle 46. The other element is bound to the particle through the element contacting the particle surface. For example, in FIG. 2B, a portion of the binding moieties 42 are bound directly to the pairing oligonucleotides 44, which are not bound directly to the particle 46. This configuration may be preferable when only one of the elements can be affixed sufficiently strongly to the particle surface, or when it is not practical to create different surface compositions on a single particle. Such conjugates of proteins and oligonucleotides are becoming widespread, and a variety of methods are available for their production; see, for example, C. M. Niemeyer et al., “Bioorganic applications of semisynthetic DNA-protein conjugates,” Chemistry—A European Journal 7:3188-3195 (2001), incorporated herein by reference. Although the capture probe 40 does not provide for the same control in the ratio of binding moiety and pairing oligonucleotide as does the capture probe 30, such control is not necessary in all applications; it may be sufficient to know the average ratio for a set of capture probes and not the ratio for each probe.

Note that by using particles to support the binding and pairing moieties, the present invention can provide orders of magnitude more binding moieties than can methods using simple protein-oligonucleotide conjugates, for the same array surface area.

When the present invention is used for multiplexed assays, the full set of capture probes contains a variety of binding moieties and a variety of pairing oligonucleotides. Unique pairs of binding moieties and pairing oligonucleotides are found on each type of capture probe, ensuring that only one type of binding moiety is targeted to an array region containing a particular capture oligonucleotide.

Binding moieties of the present invention include any moiety capable of binding to an analyte with any degree of affinity and specificity. There is essentially no limitation on the type and number of potential binding moieties that can be used in the capture probes of the present invention. Examples of binding moieties and analytes include enzymes and substrates, antibodies and epitopes, carbohydrates and lectins, receptors and ligands, and nucleic acids and complementary nucleic acids, among others Thus, binding moieties include, but are not limited to, proteins, peptides, enzymes, enzyme substrates, antibodies, antibody fragments, oligonucleotides (single-, double-, or triple-stranded DNA or RNA), oligosaccharides, hormones, opiates, steroids, hormone receptors, carbohydrates, cofactors, drugs, lectins, sugars, agonists and antagonists for cell membrane receptors, toxins and venoms, viral epitopes, and small molecules that can bind receptors or inhibit enzymes.

Binding moieties range in their affinity and specificity for analytes. High-specificity binding moieties such as protein-specific antibodies are employed in assays for particular analytes, e.g., diagnostic assays for proteins known to be markers for a particular disease. Hundreds of monoclonal and polyclonal antibodies are available commercially. Examples include antibodies to CD antigens and their receptors, histocompatibility antigens, immunoglobulin, matrix metalloproteinases and their inhibitors, and acute phase proteins. Note that antibodies can capture not only free analytes but also, in some cases, analytes with bound receptor or autoantibody.

Low-specificity binding moieties are useful for comprehensive profiling for biological marker discovery, i.e., for capturing and analyzing a large number of sample components. Captured molecules can be examined to determine those that are differentially present between subject classes, with only the relevant molecules warranting further analysis for structural identification. Class-specific capture proteins can bind a variety of proteins based on property or structure, rather than just a single protein. In some cases, it may be more efficient to capture multiple (e.g., 5-10) proteins with a single binding moiety than to develop distinct antibodies to each protein. Suitable examples of class-specific binding moieties are binding domains of proteins involved in protein-protein interactions. For example, various SH2 and PTB domains recognize multiple proteins containing phosphotyrosine. PDZ and SH3 domains can be used as stable stand-alone binding moieties; numerous distinct PDZ domains are found in scaffolding proteins, each recognizing a distinct sequence of four C-terminal residues on key signaling and other proteins. Additionally, small molecules such as ligands or cofactors can be used to capture their receptors. For example, essentially all ligands that have been used in affinity chromatography can serve as binding moieties of the capture probes of the present invention. Other examples include lectins for polysaccharides and glycoproteins, nucleic acids for nucleic acid-binding proteins, NAD for dehydrogenases, benzamides for serine proteases, and heparin for coagulation proteins.

In alternative embodiments, binding moieties capture analytes based on their biophysical properties using stationary phase chemistry. Particle surfaces are derivatized with positively-charged, negatively-charged, hydrophobic, or hydrophilic functional groups, for example, by derivatizing the particles with self-assembled monolayers terminated with carboxyl groups. The monolayers are then coupled to organic molecules bearing desired functional groups for analyte capture. Such binding moieties tend to bind to a variety of analytes in the sample, from low-molecular weight organic compounds to proteins. For example, carbohydrate-derivatized self-assembled monolayers have been used to investigate interactions between proteins and carbohydrates, as described in N. Horan et al., “Nonstatistical binding of a protein to clustered carbohydrates,” Proc. Natl. Acad. Sci. USA 96:11782-11786 (1999), incorporated herein by reference. In the present invention, carbohydrates can serve as binding moieties for other carbohydrates and also for the multiple binding determinants for carbohydrates in glycoproteins.

Stationary-phase binding moieties allow for combinatorial selection of an appropriate set of capture probes. A large variety of stationary phases are constructed, and one or more subsets are selected that maximize the information obtained from a particular biological sample with a minimal number of different capture probes. The major analytes extracted by each stationary phase are identified, and sets of capture probes providing the most efficient coverage of proteins and low-molecular weight molecules are selected for subsequent assays. Sets of capture probes can be formed by enumerating all sets of size between 1 and n, where n is the total number of different capture probes provided. One way to quantify information content provided by a capture probe set, particularly when the analyte identities are unknown, is to count the total number of distinct peaks in mass spectra obtained from analytes captured by the combined set of capture probes.

The capture probe pairing moiety is preferably a pairing oligonucleotide, but can be any member of a complementary binding pair. The pairing oligonucleotide is a single-stranded nucleotide sequence that is selected to be complementary to a surface-bound capture oligonucleotide, i.e., to have sufficient complementarity to be able to hybridize under highly stringent or mildly stringent conditions, thereby forming a stable duplex. In particular, pairing oligonucleotides include any polymeric compound capable of specifically binding to surface-bound oligonucleotides by way of a regular pattern of monomer-to-nucleoside interactions such as Watson-Crick type of base pairing, Hoogsteen or reverse Hoogsteen types of base pairing, or the like. Either the pairing or capture oligonucleotide can be modified to enhance its physical properties if desired.

The length of the pairing oligonucleotide is sufficiently large to ensure that hybridization occurs primarily with desired capture oligonucleotides and not at other sites of the array. Although any length and composition of pairing oligonucleotide and capture oligonucleotide may be employed, the length and composition are preferably optimized to allow proper self-assembly of capture probes and annealing of complementary oligonucleotides without denaturing or otherwise affecting captured analytes. For example, proteins can be denatured at the high temperatures (e.g., 65° C.) typically used to anneal oligonucleotides. Generally, high efficiency oligonucleotide binding at lower annealing temperature can be promoted by using oligonucleotides with high homology. Annealing temperatures can be lowered further by enriching for GC content. Oligonucleotides with a range of base lengths and GC content can be titrated at 40° C. to determine the optimal length and composition.

Other factors influencing the selection of oligonucleotide length include inconvenience and expense of synthesizing and purifying oligomers greater than about 30-40 nucleotides in length, the greater tolerance of longer oligonucleotides for mismatches than shorter oligonucleotides, and whether modifications to enhance binding or specificity are present. Typically, pairing oligonucleotides have lengths between about 2 and 200 nucleotides, more preferably between about 5 and 50 nucleotides, more preferably between about 5 and 20 nucleotides, and most preferably between about 13 and 17 nucleotides. Note that the number of nucleotides in the pairing oligonucleotides determines the number of different possible oligonucleotides and therefore the multiplexing level. Seventeen nucleotides provides 4¹⁷ or over 1.7×10¹⁰ different possibilities.

Oligonucleotides of the present invention are preferably synthesized by conventional means on commercially automated DNA synthesizers, preferably using phosphoramidite chemistry.

In general, oligonucleotides (pairing or capture) of the present invention may include non-phosphate internucleosidic linkages, many of which are known in the art, e.g., phosphorothioates, phosphorodithioates, phosphoramidates, peptide nucleic acids, methylphosphonates, and P-chiral linkages. Additional non-phosphate linkages include phosphoroselenoate, phosphorodiselenoate, phosphoroanilothioate, phosphoranilidate, alkylphosphotriester such as methyl- and ethylphosphotriester, carbonates such as corboxymethyl ester, carmabate, morpholino carbamate, 3′-thioformacetal, silyl such as dialkyl (C1-C6) or diphenylsilyl, sulfamate ester, and the like. Such linkages and methods for introducing them into oligonucleotides are described in, for example, Peymann and Ulmann, Chem. Rev. 90:543-584 (1990), and Milligan et al., J. Med. Chem. 36:1923-1937 (1993), incorporated herein by reference. Additional modifications such as boronated bases, cholesterol moieties, or 5-propynyl modification of pyrimidines may also be included.

When capture probes are used for in vivo experimentation, the pairing oligonucleotides are preferably unnatural oligonucleotides that resist the hydrolytic action of nucleases. Examples include peptide nucleic acids, phosphorous modifications, and other molecules with modified nucleic acid backbones, such as phosphodiesters replaced by phosphorothioates, methyl phosphonates, or sulfamate analogs.

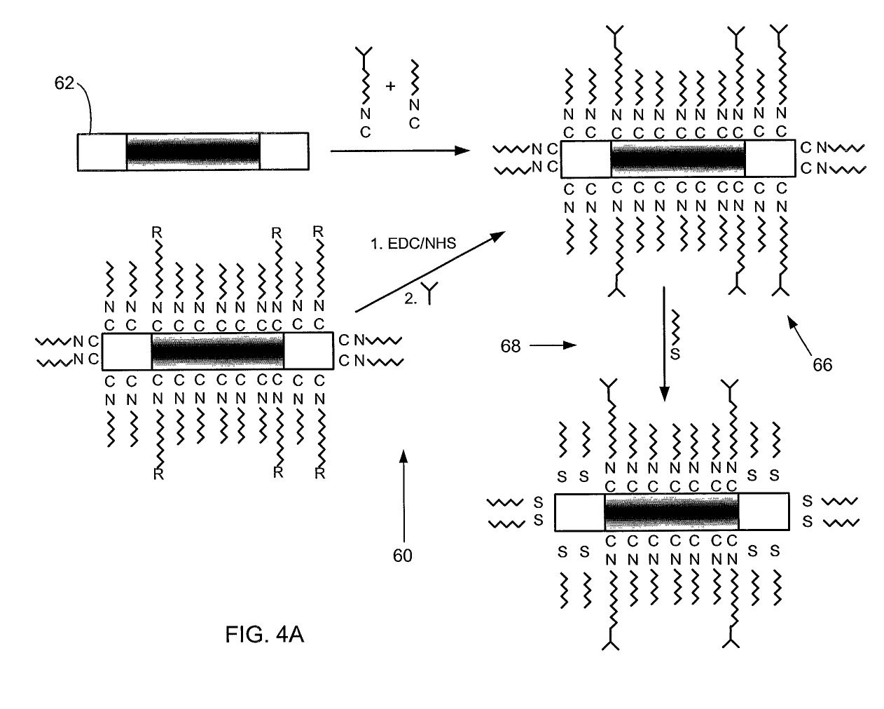

Capture probes can be constructed from any type of particle such as a latex bead. Preferably, the particles are cylindrically-shaped, segmented metal nanoparticles, as shown in FIG. 3. Suitable metals include, without limitation, gold, platinum, nickel, copper, silver, palladium, cobalt, rhodium, and iridium. The particles can also be made of a metal chalcogenide, oxide, sulfide, nitride, phosphide, selenide, telluride, or antimonide, a metal alloy, a semiconductor or semi-metal, an organic or organometallic compound or material, or a particulate or composite material. A nanoparticle 50 has all dimensions less than approximately 100 nm and contains at least two, and preferably at least three, different segments (or “stripes”) having different surface compositions. Preferred particle dimensions are between approximately 70 and 100 nm in length and between approximately 10 and 50 nm in diameter. The different surface compositions facilitate attachment and localization of the binding moieties and the pairing oligonucleotides to different regions of the surface. In FIG. 3, the binding moieties are fixed to an inner region 52, and the pairing oligonucleotides to outer regions 54 and 56. Although it is preferred that the binding moieties and pairing oligonucleotides remain in separate regions to prevent the binding moieties and captured analytes from sterically interfering with oligonucleotide hybridization, it is not necessary. For the same reason, it is preferred that the outer regions 54 and 56 contain the pairing oligonucleotides, with the binding moieties localized to the inner region 52.

The ratio of the surface area of the different segments 52, 54, and 56 determines (in part) the number of analyte molecules that can be captured by a single capture probe 50. The surface area of a particular segment can be increased either by increasing the segment length or by adding roughness or porosity to the segment.

In order for the capture probes to self-assemble to the correct location on the array of surface-bound capture probes, non-specific binding of oligonucleotides to the particle should be minimized. Latex particles tend to have a higher degree of non-specific binding than do metal particles. In addition, micron-sized particles tend to have roughness on the scale of many nanometers. Because the oligonucleotides have lengths on the order of less than ten nanometers, the scale disparity between a large particle and oligonucleotide tends to preclude highly specific binding, which is necessary for accurate self-assembly.

In one embodiment of the invention, one of the different regions 52, 54, and 56 is made of a ferromagnetic material such as cobalt or nickel. Ferromagnetic materials with characteristic dimensions of tens of nanometers or less exhibit superparamagnetic behavior; both Co and Ni have been shown to be superparamagnetic at dimensions of 8 nm. Superparamagnetic materials do not retain their magnetism in the absence of a magnetic field at room temperature. Nanoparticle segments of dimensions at most approximately 10 nm render the particle superparamagnetic. As a result, the particles can be collected by applying a magnetic field and then redispersed upon removal of the field. Because the segments must be very short to render the particles superparamagnetic, they typically do not provide sufficient surface area for supporting either the binding moieties or the pairing oligonucleotides. Preferably, magnetic capture probes contain at least four different segments, e.g., Au/Pt/Ni/Au, of which the nickel (or cobalt) segment provides magnetic properties but is not necessarily derivatized.

Magnetic particles enable three specific applications. First, magnetic nanoparticles applied to cells or mixed sera samples can be removed by applying a magnetic field, providing a particle separation mechanism, or at least eliminating the need to centrifuge the sample. Additionally, superparamagnetic particles self-agitate in the presence of a spatially-varying magnetic field, acting as miniature stir bars. Self-agitation obviates the need for external agitation during incubation and wash steps. Third, the transport of magnetic particles to a hybridization surface can be accelerated by applying a magnetic field.

The particles of FIG. 3 are advantageous because their stripe pattern serves as a nanoscale barcode that can be used to encode the identity of the attached binding moieties and pairing oligonucleotides. This may be useful to confirm that the capture probes self-assemble to the correct locations or to identify the probes after they are removed from the array. In this case, each type of capture probe contains a different stripe pattern. Based on the different wavelength-dependent reflectivities of different metals, the particles can be decoded by straightforward optical microscopy techniques. For more information on this usage of the capture probes, see U.S. patent application Ser. No. 09/677,198, “Colloidal Rod Particles as Nanobar Codes,” filed Oct. 2, 2000, incorporated herein by reference.

The cylindrical nanoparticles are preferably prepared by electrochemical deposition of metal ions in solution into template pores. Preferably, the template is a membrane with essentially linear pores over the entire membrane thickness. Suitable template materials include polycarbonate and alumina. Polycarbonate membranes with a variety of pore diameters less than 100 nm are commercially available, e.g., Nuclepore® polycarbonate Track-Etch membranes, available with diameters of 15, 30, 50, 80, and 100 nm. Polycarbonate membranes typically have lower pore densities than do alumina membranes, yielding fewer particles per synthesis. Alumina membranes with pore diameters of less than 100 nm are not available commercially and must be prepared. Suitable preparation methods are described in C. R. Martin et al., “Nanomaterials: A Membrane-Based Synthetic Approach,” Science 266:1961-1966 (1994); C. A. Foss et al., “Template-Synthesized Nanoscopic Gold Particles: Optical Spectra and the Effects of Particle Size and Shape,” J. Phys. Chem. 98:2963-2971 (1994); M. El-Kouedi et al., “Electrochemical Synthesis of Asymmetric Gold-Silver Iodide Nanoparticle Composite Films,” Chem. Mater. 10:3287-3289 (1998); H. Masuda et al., “Square and Triangular Nanohole Array Architectures in Anodic Alumina,” Adv. Mater. 13:189-192 (2001); and T. Thrun-Albrecht et al., “Ultrahigh-density nanowire arrays grown in self-assembled diblock copolymer templates,” Science 290:2126 (2000), all incorporated herein by reference.

Methods for manufacturing micron-sized cylindrical nanoparticles are described in U.S. patent application Ser. No. 09/969,518, “Method of Manufacture of Colloidal Rod Particles as Nanobarcodes,” filed Oct. 2, 2001, incorporated herein by reference. In the present invention, similar methods are employed to manufacture nanoparticles with dimensions of at most approximately 100 nm. Briefly, silver is evaporated onto one side of a porous alumina or polycarbonate membrane, allowing it to be used as a cathode. The silver solution is then removed and replaced with a second metal solution, which is electroplated onto the silver for a desired length of time. The process is repeated with different solutions until the desired number of segments has been produced. Any suitable metals and solutions can be used. The segment length is controlled by varying the number of Coulombs passed, which can be monitored accurately. Particles containing Ni or Co may require different electroplating conditions or shorter exposure times. After the particles reach the desired length and segment number, the silver backing and alumina or polycarbonate membrane are dissolved using acid, base, or methylene chloride, respectively, leading to a freestanding suspension of particles. Additional steps may be necessary to remove residual alumina or polycarbonate from the particle surfaces, e.g., by brief exposure to H₂SO₄/H₂O₂ (for Au/Pt particles) followed by centrifugation and resuspension in water. Root-mean-square roughness at the interface between segments is preferably no greater than 5 nm. Stripe roughness can be determined by high resolution transmission electron microscopy (TEM) or field emission-scanning electron microscopy (FE-SEM) or by atomic force microscopy (AFM) of particles still attached to the Ag electrode but with the template membrane dissolved. If necessary, electropolishing steps can be carried out between metal depositions to ensure smooth interfaces.

If desired, the particles can be characterized using methods such as near-field scanning optical microscopy (NSOM). Because of its extremely high resolution (1 nm), NSOM can be used to probe the chemical composition and physical structure of segment interfaces. AFM and FE-SEM can also be used. In these methods, the particles are covalently tethered to optical-quality glass slides modified with 3-amino- or 3-mercapto-propyltrimethoxysilane. For TEM, particle suspensions in water are drop-coated onto standard TEM grids. Traditional bulk methods can also be used to characterize the physical properties and chemical functionality of the particles. For example, the discrete dipole approximation can be used to generate accurate descriptions of the optical properties of complex-shaped colloidal and Ag nanostructures.

Attachment of binding moieties and pairing oligonucleotides to the particles is preferably performed so that each entity is bound primarily to its desired location on the particle surface. For example, for gold and platinum particles, 90% of the binding moiety is preferably restricted to the platinum surface, and 90% of the pairing oligonucleotide is preferably restricted to the gold surface. This is facilitated by the different compositions of the surface of each segment. For example, DNA can be attached to gold surfaces and antibodies to platinum surfaces. Nickel surfaces can be derivatized with either reagent. One synthetic scheme is based on the fact that isonitrile groups (RNC) adsorb irreversibly to platinum, but can be displaced from gold by large concentrations of short-chain thiols, which in turn can be displaced by low concentrations of longer-chain oligonucleotide-functionalized thiols. When the particle contains a nickel segment, pyridine, histidine, and glyoxime derivatives, which have high selectivity for nickel, can be attached to nickel and easily displaced from other metals.

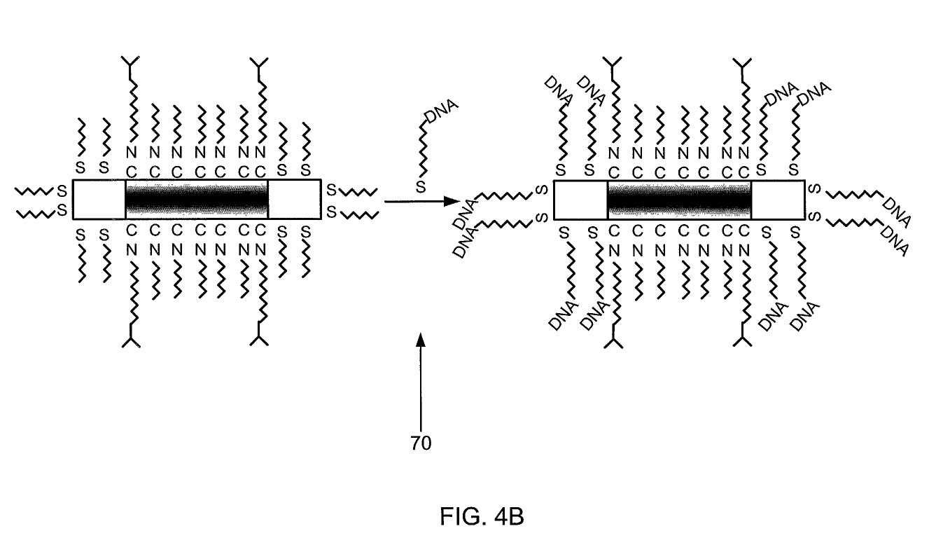

FIGS. 4A and 4B illustrate two possible methods using this scheme to derivatize nanoparticles with proteins and oligonucleotides. The procedure is general and can be used to attach any protein and oligonucleotide sequence to a particle. A first step 60 yields an intermediate 66 by one of two methods. In the first such method, a protein (the binding moiety) is directly coupled to a carboxyl-terminated alkylisocyanide (RNC, where R is HO₂CX, with X a medium-chain alkyl or aryl group) via 1-ethyl-3-(3-dimethylaminopropyl) carbodiimide (EDC) and N-hydroxyl sulfosuccinimide (NHS) activation, or by reaction of the carboxylate with trifluoroacetic acid to form an anhydride. Many biological molecules contain amines, which can be conjugated easily to carboxylates using this method. To prevent polymerization in future steps, only one or two isocyanides are affixed to each protein. Underivatized particles 62 having platinum and gold segments are reacted with the protein and with alkyl isocyanide to form the intermediate 66, a particle with a mixed monolayer. Dilution with alkyl isocyanide is typically necessary to form uniform monolayers. The intermediate 66 can alternatively be formed by forming a mixed layer of isocyanides on the particle, activating the monolayer with EDC/NHS, covalently attaching binding moieties, and then quenching unreacted, activated sites with ethanolamine. The monolayer chain length can be optimized based on the analyte-binding moiety and other relevant conditions. Because single-stranded oligonucleotides have primary amines (exocyclic amines) that are susceptible to ester chemistry, it is important to conjugate the carboxy monolayer to the amine-containing molecules before introducing the oligonucleotide to avoid detrimental derivatization of the oligonucleotides.

To minimize the role of steric or ionic interactions in capturing analyte, the monolayer bearing the binding moiety can be derivatized with bifunctional crosslinkers to increase the distance between the metal surface and the binding moiety. For example, a thin layer of polymeric material can be built up between the nanoparticle surface and binding moieties. Multiple materials have been used for this purpose, such as polylysine, aminodextran, and streptavidin. The additional layer increases the number of functional groups available on the surface for further derivatization, minimizes the nonspecific interactions between the metallic surface and biological molecules, extends the binding moieties away from the surface to decrease the steric hindrance of analyte capture, and helps molecules maintain their original bioactivity by functioning as a soft interface between the rigid particle surface and the binding moieties. The layer therefore increases detection sensitivity while lowering the number of particles required per assay.

In a second step 68, the intermediate 66 is reacted with a short-chain organothiol, which displaces the isocyanates bearing binding moieties from the gold surfaces. In a third step 70 (FIG. 4B), low concentrations of thiolated oligonucleotides replace the short-chain alkylthiols on the gold surfaces. The length and density of attached oligonucleotides can be optimized for a particular assay.

As will be apparent to those of skill in the art, other methods may be employed to attach the binding moieties and capture oligonucleotides to distinct regions of the particle surface. For example, the ends of the particles can be derivatized with oligonucleotides before being removed from the template. After template dissolution, the center region of the particle can be derivatized with the binding moiety. Alternatively, the particles can be made with a magnetic segment, coated with pairing oligonucleotides, and combined with small magnetic beads coated with the binding moiety. The small magnetic beads then associate with the particle.

If desired, bulk methods can be used to verify that the pairing oligonucleotides and binding moieties have been attached correctly. For example, detection of emission from centrifuged and resuspended particles containing attached fluorescently-labeled proteins or DNA can verify that attachment has occurred. The use of two fluorophores with different emission characteristics verifies attachment of both biomolecules. Characterization of the spatial fidelity of functionalization may require particle immobilization, which can be accomplished by EDC/NHS activation of carboxylates on the bound proteins and exposure to amino-functionalized glass slides. NSOM and AFM can generate nanometer-scale chemical functionality maps of the immobilized particles by exploiting the molecular recognition properties of the surface-confined molecules. For NSOM experiments, particles with fluorescently-labeled complementary DNA or analyte can be reacted and the fluorescence recorded as a function of position. For AFM, DNA or analyte tagged with colloidal gold particle can be used, and topography measured as a function of position.

The array of complementary surface-bound capture oligonucleotides is composed of different capture oligonucleotide sequences localized to particular regions of the surface. Capture probes are directed to regions containing capture oligonucleotides complementary to their pairing oligonucleotides.

Typically, the pairing oligonucleotide and capture oligonucleotide that form a binding pair have sequences that are completely complementary. However, absolute (100%) complementarity is not necessary for self-assembly of capture probes, particularly in the case of longer oligonucleotides. In general, any oligonucleotides that are hybridizable, i.e., that form a stable duplex or binding complex, are suitable; this condition is referred to herein as “substantially complementary.” Generally, the larger the oligonucleotides, the larger the number of mismatches that can be tolerated. More than one mismatch may not be suitable for oligonucleotides of less than about 21 nucleotides. One skilled in the art may readily determine the degree of mismatching that can be tolerated between any two oligomers based on the thermal stability of the resulting duplex, as measured by its melting point using standard techniques.

Arrays containing position-addressable surface-bound capture oligonucleotides are available commercially or can be prepared using methods known in the art. Preferably, the arrays are produced through spatially-directed oligonucleotide synthesis, which includes any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations, and sequestration with physical barriers. In general, these methods involve generating active sites, usually by removing protective groups, and coupling to the active site a nucleotide that, itself, optionally has a protected active site if further nucleotide coupling is desired. Oligonucleotide array synthesis methods are described in V. G. Cheung et al., “Making and reading microarrays,” Nature Genetics Suppl. 21:15-19 (1999), and R. J. Lipshutz et al., “High density synthetic oligonucleotide arrays,” Nature Genetics Suppl. 21:20-24 (1999), both incorporated herein by reference. See also U.S. Pat. No. 5,143,854, issued to Pirring et al., U.S. Pat. No. 5,571,639, issued to Hubbell et al., U.S. Pat. No. 5,624,744, issued to Sundberg et al., and U.S. Pat. No. 5,412,087, issued to McGall et al., all incorporated herein by reference, and the references cited therein.

Solid substrates for supporting the capture oligonucleotides can be biological, nonbiological, organic, inorganic, polymeric, or a combination of these. The surface is preferably flat but can take on alternative surface configurations, e.g., have depressed or raised regions. If necessary, the substrate can be chosen to provide appropriate light-absorbing characteristics. Suitable substrate materials include functionalized glass, Si, Ge, GaAs, GaP, SiO₂, SiN₄, modified silicon, or a gel or polymer substrate. Preferably, the surface of the solid substrate contains reactive groups such as carboxyl, amino, hydroxyl, or thiol. Preferably, the surface is optically transparent and has surface Si—OH functionalities, such as found on silica surfaces. The surface can also be porous. If the array is interrogated directly by laser desorption, the substrate can be a gold MALDI plate.

The substrate area devoted to a particular capture oligonucleotide can depend upon a variety of factors including the sample volume and nature, detection technique, multiplexing level of the assay, and number of different capture probes provided. In general, it is preferred that a single array of capture oligonucleotides be designed for use in multiple different types of assays, and thus not tailored to a particular sample and assay. Additionally, it is preferred that the array be reusable. One suitable configuration is a standard 50 mm×50 mm MALDI slide, which accommodates 800 5×5 spot microarrays, each having 200 μm-diameter spots separated by 200 μm.

Surface-bound oligonucleotide arrays of the present invention typically include between about 5×10² and about 10⁸ oligonucleotides per square centimeter, or between about 10⁴ and about 10⁷, or between about 10⁵ and 10⁶ oligonucleotides per square centimeter.

When a multiplexed assay of the present invention is performed, any number of different capture probes are incubated first with the sample, and then preferably recovered from solution. The capture probes and array are incubated for a desired period of time at the desired temperature, and the array is then washed to remove unbound capture probes, leaving an array of capture probes hybridized to capture oligonucleotides. Optimal hybridization conditions depend upon the nature and length of the oligonucleotides, as discussed above, and the characteristics of the binding moieties and analytes. Preferably, greater than 95% accuracy of self-assembly is achieved; that is, fewer than one in twenty capture probes bind to the incorrect address.

In some embodiments, a large number of capture probes is combined with the sample, and insufficient array surface area is provided for their capture. Additional dimeric oligonucleotides can be introduced that are capable of hybridizing to pairing oligonucleotides on two different capture probes simultaneously, resulting in an aggregate of capture probes. This linker oligonucleotide is added to the hybridization buffer to crosslink capture probes and to provide a seed driving aggregation of capture probes at a particular location. The aggregate binds to the array to coat it with more than a monolayer of capture probes. Selective, DNA-driven aggregation of colloidal gold nanoparticles in solution is described in detail in C. A. Mirkin et al., “A DNA-based method for rationally assembling nanoparticles into macroscopic materials,” Nature 382:607-609 (1996); R. Elghanian et al., “Selective colorimetric detection of polynucleotides based on the distance-dependent optical properties of gold nanoparticles,” Science 277:1078-1080 (1997); J. J. Storhoff et al., “One-Pot Colorimetric Differentiation of Polynucleotides with Single Base Imperfections Using Gold Nanoparticle Probes,” J. Am. Chem. Soc. 120:1959-1964 (1998); and C. A. Mirkin et al., PCT Published Application Nos. WO 98/04740 and WO 97/12783, all incorporated herein by reference.

Aggregates of capture probes can be removed from solution before being exposed to the array. In this embodiment, the linker oligonucleotide dimer is added to the sample containing capture probes, and the aggregates are separated by, e.g., centrifugation. Different types of capture probes can be selectively and sequentially aggregated. A first linker dimer is added to the solution to aggregate one type of capture probe, and the aggregate is recovered from solution. Next, a second linker dimer is added to the solution to aggregate capture probes bearing a different pairing oligonucleotide. This aggregate is then removed. The process can be repeated for as many different capture probes as desired.

In one embodiment of the invention, instead of creating a direct linkage between the pairing oligonucleotide and capture oligonucleotide, immobilization is initiated using a third oligonucleotide sequence that is complementary to both capture and pairing oligonucleotides. Each capture probe to be immobilized has a unique dimeric linker strand that possesses a generic sequence complementary to the pairing oligonucleotide and a unique sequence complementary to the capture oligonucleotide. In this case, the oligonucleotide of the capture probe and the linker are referred to as a double-stranded pairing oligonucleotide. While this embodiment requires an additional step of incubating each capture probe type with its linker strand, capture probe synthesis is simplified because the identical oligonucleotide is affixed to each capture probe. This embodiment also provides additional flexibility by allowing both perfect segregation of each type of capture probe as well as arbitrary grouping of probes. For example, a single-spot assay can be developed for five different analytes by using the same linker strand to target five different binding moieties to the same location of the array. This embodiment also allows capture probes of interest to be manufactured independently of the array. This “three-strand” approach has been applied by Mirkin et al. as described in the references cited above.

In a preferred embodiment, captured analytes are detected by mass spectrometry, preferably using matrix-assisted laser desorption ionization time-of-flight (MALDI-TOF) mass spectrometry. In one embodiment, the substrate supporting the capture oligonucleotides is a gold MALDI plate, and bound analyte is not removed from the plate before being interrogated. After capture probe self-assembly, the plate is quickly washed, dried, and sprayed with a matrix solution before being subjected to MALDI-TOF. Alternatively, the bound analyte (and possibly also the capture agent) is transferred to a replica plate (e.g., nitrocellulose) in a manner that retains the spatial distribution of capture probes. In either case, a laser is focused at each address location having the same bound capture probe for desorption and ionization of analytes. Mass spectra from individual laser shots in each array location are averaged. Typically, between approximately 50 and 500 laser shots are averaged at each location. If desired, an intelligent algorithm can be applied to select and average only particular spectra. The resulting spectra can then be correlated with each location to quantify or confirm the identity of analyte captured by a high-specificity capture probe or to determine the structure of an analyte captured by a low-specificity capture probe. If desired, higher molecular-weight analytes can be removed from the array and digested for analysis by liquid chromatography-tandem mass spectrometry (LC-MS/MS). Further analysis can be applied to the acquired spectra as desired.

The surface area of the array devoted to a particular capture oligonucleotide can be selected to optimize the MALDI signal. Conventional MALDI instruments have a laser spot size of approximately 100 μm in diameter. For capture probes bearing approximately 200 analytes per probe, and assuming 10⁶ capture probes per spot, femtomole amounts of analyte can be interrogated by a single laser spot. This amount of analyte is within the detection limits of current MALDI instruments. This configuration reduces the need for multiple sampling on larger areas by the laser, thereby precluding the search for MALDI hotspots and increasing the MALDI readout throughput. In assays for analytes present in low concentrations, the capture spot size can be increased to accommodate larger numbers of a given type of capture probe.

Note that oligonucleotides have very poor ionization efficiencies and require specific matrices to generate acceptable ionization efficiencies. Commonly-used matrices for peptides and proteins do not provide efficient oligonucleotide ionization, and significant interference from the oligonucleotides in the mass spectra should not occur.

When the capture probes are made of metal particles, the particles can be interrogated directly by mass spectrometry and, as determined by the present inventors, generally provide a higher signal-to-noise ratio than capture probes incorporating latex particles or without particles. For more information on this phenomenon, see U.S. patent application Ser. No. 09/920,440, “Methods for Solid Phase Nanoextraction and Desorption,” filed Aug. 1, 2001, incorporated herein by reference. One reason for the improved signal is that the particles concentrate analyte to a small area. Additionally, increased electromagnetic field strength and rapid heat transfer at the particle surface enhance the ionization efficiency. These features can be exploited by maximizing the overlap of the laser spectrum and the surface plasmon absorption features of the metal supporting the analyte-binding moieties. Most commercial MALDI systems use N₂ lasers with a peak emission at 337 nm, but other types of lasers can be employed; examples include multi-wavelength gas lasers such as krypton-argon lasers, tunable dye lasers, or tunable solid state lasers such as titanium sapphire lasers.

In some cases, it is desirable to remove the bound analyte, with or without capture probes, from the array before acquiring mass spectra. Eluted analytes can be analyzed by MALDI-TOF or electrospray ionization (ESI) MS. A variety of methods can be employed to remove the analyte from the array of capture oligonucleotides. In one embodiment, the array is heated above the oligonucleotide melting point to release the capture probe with bound analyte. It can also be heated to denature the captured analyte or binding moiety. By using a laser with a suitable spot size to heat the array locally, different analytes can be removed independently. Alternatively, the capture probe can include a cleavable linker between the particle and binding moiety. Suitable linkers are available that are cleavable by addition of photons, change of temperature, or other suitable energy input. In one embodiment, either the binding moiety or pairing oligonucleotide is covalently bound to the calcium-binding protein calmodulin (CaM), while the particle is derivatized with a CaM-binding protein or sequence. The capture probe is maintained in a solution containing calcium ions (Ca²⁺). In the absence of Ca²⁺, CaM dissociates from the CaM-binding sequence to release the binding moiety and bound analyte for transfer to a replica plate for further analysis. Dissociation can be induced by adding the calcium chelator ethyleneglycol bis(β-aminoethyl ether)-N,N,N′,N′-tetraacetic acid (EGTA) to the array. Alternatively, a photolabile amino acid can be included in the binding moiety; upon irradiation, the linkage is cleaved. When the analytes are to be detected by MALDI-TOF, dissociation may be induced by applying an acidic matrix material to the array.

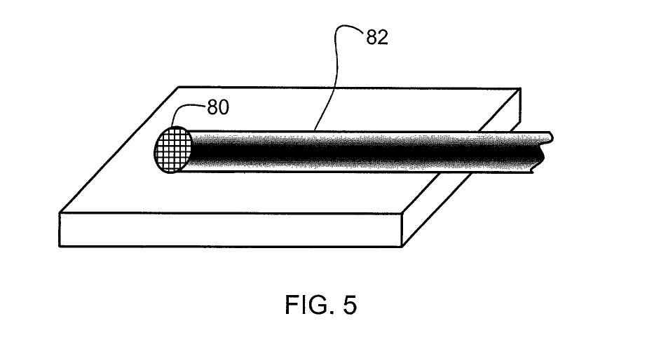

FIG. 5 illustrates an embodiment of the invention in which the array of surface-bound capture oligonucleotides is coupled to an elution device. In this case, the array 80 is part of a microfluidic device containing a flow channel 82. After the capture probes are incubated with the array, a suitable solvent is introduced. The solvent removes captured analyte from the array, and the resulting solution flows through the channel by capillary action. The solution can then be directed to a detection mechanism for further analysis. As will be apparent to those of skill in the art, the present invention can be coupled to a variety of suitable sample handling or detection devices.

If desired, an additional detection mechanism can be employed to read the stripe pattern on each capture probe to identify the binding moiety.

While mass spectrometry is a preferred detection method because of the structural information it provides, the present invention can be practiced with alternative detection methods. These methods may involve tagging the captured analyte, and include all luminescent methods such as fluorescence, chemiluminescence, or electroluminescence; spectroscopies such as absorbance, Raman, and surface plasmon resonance; and radioactivity, as well as enzymatic signal amplification. For example, surface plasmon resonance has been used to detect binding to DNA arrays, as described in B. P. Nelson et al., “Surface Plasmon Resonance Imaging Measurement of DNA and RNA Hybridization Adsorption onto DNA Microarrays,” Anal. Chem. 73:1-7 (2001), incorporated herein by reference.

The present invention can be used for analysis of any complex biological sample, including blood, urine, cerebrospinal fluid, cells, and tissue, among others. Thousands of capture probe types can be added to a small sample volume. In fact, since the capture probes are at most 100 nanometers in length, they can be inserted into cells or into the bloodstream of animals and then recovered. Depending upon the type of sample, preliminary separation steps can be performed before contact with the capture probes of the present invention.

The present invention can be used to perform any type of multiplexed assay required, such as diagnostic assays for multiple antigens known to be indicative of a particular disease or other physiological condition. The invention is particularly useful for detecting analytes at very low concentrations, which may not be detected using conventional tools such as protein arrays. One important application of the invention is differential phenotyping for biological marker discovery, in which a large number of analytes are detected simultaneously in a single biological sample. Biological markers, or biomarkers, are measured characteristics of a subject that are indicative of normal or pathological biological processes, response to therapy, or other clinical endpoints. By collecting samples from, e.g., healthy and diseased patients, drug responders and non-responders, or the same patients at different time points, differences in levels of particular analytes can be found that are indicative of the condition being investigated. While the large majority of measured analytes exist at comparable levels in both groups of subjects or time points, some analytes have statistically significantly different values between the two groups, and these analytes may serve as diagnostic or other biomarkers. In biomarker discovery studies, because the analyte of interest is unknown, it is important to be able to measure as many analytes as possible from a small sample volume so that relevant analytes or patterns of analytes can be identified. The present invention is advantageous because it provides a high level of multiplexing and can identify analytes often missed using other techniques.

Additionally, the present invention is useful for identifying low-molecular weight species with unknown structure. For example, if the mass spectrum of array location X has a peak that is of significantly different intensity in, e.g., diseased versus healthy subjects, an efficient route to its absolute structural determination is to acquire a larger amount of the analyte using the chemistry of the particle assembled at location X, but on a much larger scale. With solution-phase capture in which the analyte is eluted from the particle before structural analysis, it would not be possible to scale up the chemistry, because the relevant binding moiety would be unknown. With the present invention, however, it is straightforward to determine which capture probe must be produced and in what amount.

The disclosure of every patent and publication referred to herein is incorporated by reference herein in its entirety.

It should be noted that the foregoing description is only illustrative of the invention. Various alternatives and modifications can be devised by those skilled in the art without departing from the invention. Accordingly, the present invention is intended to embrace all such alternatives, modifications and variances that fall within the scope of the disclosed invention.

What is claimed is:

1. A method for detecting an analyte in a sample suspected of containing said analyte, comprising:
a) contacting said sample with a plurality of capture probes, each capture probe comprising a particle, at least one binding moiety, and at least one pairing oligonucleotide, wherein said binding moiety is capable of binding to said analyte;
b) after step (a), contacting said capture probes with a solid support having an array of surface-bound capture oligonucleotides, wherein at least one of said surface-bound capture oligonucleotides is substantially complementary to at least one of said pairing oligonucleotides, whereby said at least one complementary pairing oligonucleotide and said at least one surface-bound capture oligonucleotide hybridize to form a binding complex; and
c) detecting analytes bound to said binding complex.

2. The method of claim 1, wherein different surface-bound capture oligonucleotides have different sequences, and wherein particular sequences are located at particular positions on said solid support.

3. The method of claim 2, wherein said particular positions are predetermined positions.

4. The method of claim 1, wherein said particle has dimensions of at most approximately 100 nm.

5. The method of claim 1, wherein said particle is made of at least one metal.

6. The method of claim 1, wherein said particle has at least two different segments, and wherein said binding moiety and said pairing oligonucleotide are affixed to different ones of said segments.

7. The method of claim 1, wherein different subsets of capture probes have different pairing oligonucleotides and different binding moieties.

8. The method of claim 7, wherein said array comprises different surface-bound capture oligonucleotides substantially complementary to said different pairing oligonucleotides.

9. The method of claim 1, wherein said pairing oligonucleotide is a double-stranded oligonucleotide.

10. The method of claim 1, wherein said binding moiety is a protein.

11. The method of claim 1, wherein step (c) comprises removing said bound analytes from said array.

12. The method of claim 1, wherein said binding moiety is capable of binding to a plurality of different analytes.

13. The method of claim 1, wherein said bound analytes are detected by mass spectrometry.

14. A method for detecting analytes in a sample suspected of containing said analytes, comprising:
a) contacting said sample with a plurality of subsets of capture probes, each capture probe in a particular subset comprising a particle, a particular binding moiety, and a particular pairing moiety, wherein each particular binding moiety is capable of binding to one of said analytes;
b) after step (a), contacting said subsets of capture probes with a solid support having an array of different complementary surface-bound moieties at particular locations on said surface, wherein said complementary surface-bound moieties are capable of binding to said pairing moieties, whereby said pairing moieties and said complementary surface-bound moieties form binding complexes; and
c) detecting analytes bound to said binding complexes.

15. A capture probe comprising:
a) a particle having at least two segments and dimensions of at most approximately 100 nm;
b) a plurality of binding moieties affixed to at least one of said segments; and
c) a plurality of oligonucleotide sequences affixed to at least one of said segments.

16. The capture probe of claim 15, wherein said particle is a cylindrical particle.

17. The capture probe of claim 15, wherein said particle is a metal particle.

18. The capture probe of claim 15, wherein said binding moiety is a protein.

19. The capture probe of claim 15, wherein said binding moieties and said oligonucleotides are affixed to different ones of said segments.

20. The capture probe of claim 15, wherein at least one of said segments is made of a ferromagnetic material.
